(19) United States
(12) Patent Application Publication
(10) Pub. No.: US 2005/0260661 A1
(43) Pub. Date: Nov. 24, 2005

(54) Assays for the identification of modulators of MHC class II expression

(76) Inventors: Bernard Mach, Chambesy (CH); Krzysztof Masternak, Morges (CH); Nicolas Fischer, Geneva (CH)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/123,269

(22) Filed: May 5, 2005

Related U.S. Application Data
(60) Provisional application No. 60/568,565, filed on May 5, 2004.

(51) Int. Cl. C12Q 1/68; G01N 33/574

(52) U.S. Cl. 435/6; 435/7.23

(57) ABSTRACT

The present invention provides methods for accurate and efficient detection of compositions that specifically modulate the interaction and/or activity of CIITA, RFXANK, RFXAP and RFX5 polypeptide factors. Further methods are provided for identifying compositions whose specific modulation of these transcription factors affects expression of a MHC class II promoter. The disclosed methods are configured in an assay format useful in moderate and high throughput screening applications.

### RELATED APPLICATIONS

This application claims the benefit of U.S. Provisional Application No. 60/568,565, filed May 5, 2004, which is incorporated herein by reference in its entirety.

### FIELD OF THE INVENTION

The invention relates to methods for the identification of compounds, that modulate (e.g., inhibit or enhance) MHC class II expression. More specifically, the compounds modulate the activity of proteins and other factors involved in MHC class II promoter activation. The invention also relates to compositions containing compounds identified using these methods, and their use for treating and alleviating symptoms associated with diseases associated with aberrant MHC class II expression, or as immunosuppressive agents.

### BACKGROUND OF THE INVENTION

MHC-II expression is principally regulated at the level of transcription. Transcription from the MHC-II promoters requires four specific proteins (besides other more general transcription factors): namely, RFXAP, RFXANK, RFX5 and CIITA. Mutations that abolish transcription have been described in each of these factors, thereby demonstrating the absolute requirement of these factors for MHC-II expression.

RFXAP, RFXANK and RFX5 (the “RFX proteins”) form a complex (the “RFX complex”) that binds the MHC-II promoter. The association of RFX complex with the promoter is stabilized by two other transcription factors, X2BP (CREB) and NF-Y, which bind to nearby sites. CIITA does not appear to bind DNA by itself but is recruited to the MHC-II promoter by multiple protein-protein interactions with the DNA platform formed by the RFX complex, X2BP (CREB), and NF-Y bound to the promoter/RFX complex and with other transcription factors bound on the promoter. CIITA interacts with downstream components of the general transcription machinery to activate transcription.

While the role of CIITA, RFXANK, RFXAP and RFX5 in MHC class II expression has been demonstrated, researching the biology of these polypeptide factors continues to be an intensive field of study. Several methods have been used to study the protein-protein interactions between the RFX proteins that form a complex, as well as between the RFX proteins and CIITA. These methods include immunoprecipitations, GST-pulldowns and promoter pulldowns. However, the identification of CIITA and/or RFX inhibitors currently requires cellular assays to confirm activity. These methods are time-intensive and not well-suited to medium or high throughput screening assay approaches. The lack of a direct in vitro assay for detecting the interactions between single RFX protein and CIITA, between a complex of RFX proteins and CIITA, as well as detecting the binding of these proteins, alone in combination, to DNA has been a major limitation to identifying inhibitors of these interactions.

Accordingly, there exists a need to identify compounds and other agents that modulate (e.g., reduce) the activity of CIITA, RFXANK, RFXAP and RFX5, which, in turn, will modulate (e.g., repress) MHC class II expression. In addition, there exists a need to identify compounds and other agents that modulate (e.g., disrupt) the protein-protein interactions between the three RFX proteins, as well as the interactions between the RFX complex and CIITA. There is also a need to identify compounds and other agents that modulate the interaction between these transcription factors and DNA.

### SUMMARY OF THE INVENTION

The invention provides methods of identifying agents or compounds that modulate, e.g., inhibit or enhance MHC II gene expression. More specifically, the invention provides methods of identifying compounds that modulate MHC II promoter activity.

A compound that modulates MHC II gene expression is identified by contacting a test cell containing a first nucleic acid having a first reporter gene linked, e.g., operably linked, to an MHC II promoter or fragment thereof with a test compound. The amount of reporter gene expression in the test cell is compared to the amount of reporter gene expression in a control cell that has not been contacted with the test compound. An increase or a decrease of the reporter gene expression in the test cell compared to the control cell indicates that the test compound is a modulator of MHC II gene expression. Preferably, the test cell contains the nucleic acid having a reporter gene linked to an MHC II promoter or fragment.

Optionally, the test cell further contains a second nucleic acid containing a second reporter gene linked to a non-MHC II promoter, e.g., an interferon-γ promoter. The first reporter gene and the second reporter gene are different. The cell is further contacted with an inducer compound. An inducer compound is a compound that is capable of activating both the MHC-II promoter and the non-MHC II promoter. For example, when the non-MHC II promoter is an interferon-γ promoter, the inducer compound is interferon-γ. The amount, e.g., level of expression of the first reporter gene is compared expression of the second reporter gene. A decrease in expression of the first reporter gene compared to the second reporter gene indicates that the test compound is an inhibitor of MHC II gene expression. In contrast, an increase of expression of the first reporter gene compared to the second reporter gene indicates that the test compound is an enhancer of MHC II gene expression.

The cell transiently expresses the nucleic acid. Alternatively, the nucleic acid is stably integrated into the cell genome. The cell is any cell capable of expressing the nucleic acid. For example, the cell is a myeloma cell. An MHC II promoter includes, for example, HLA-DRA (i.e., HLA-DRA1); HLA-DRB (i.e., HLA-DRB1); HLA-DPA1; HLA-DPB1; HLA-DQA1; HLA-DQB1. An MHC II promoter fragment is a functional fragment. By functional fragment is meant the fragment is capable of inducing MHC II gene expression. For example, the MHC II promoter fragment contains and X box, X2 box, Y box or W box sequence or combination thereof.

The reporter gene encodes a protein that produces a detectable signal. For example, the reporter gene encodes for a bioluminescent protein such as ferredoxin IV, green fluorescent protein, red fluorescent protein, cyan fluorescent protein, yellow fluorescent protein, blue fluorescent protein, the luciferase family and the aequorin family, the phycobiliproteins (including B-Phycoerythrin (B-PE), R-Phycoerythrin (R-PE), and Allophycocyanin (APC)), Aequorin, obelin, or bacterial lux bioluminescence genes from Vibrio fischeri.

A compound that modulates MHC II gene expression is identified by bringing into contact an MHC II promoter or fragment thereof, a RFX complex and a test compound under conditions where the MHC II promoter or fragment thereof, and the RFX complex, in the absence of compound, are capable of forming a complex. Optionally, a CIITA polypeptide and or a X2BP polypeptide and a NF-Y polypeptide are brought in contact with MHC II promoter or fragment thereof, a RFX complex and test compound. An RFX complex includes a RFXAP, RFXANK and RFX5 protein. The MHC II promoter, or the CIITA polypeptide is immobilized solid support, e.g., bead, plastic, paper, glass, or chip. Alternatively, the MHC II promoter, the CIITA polypeptide, the RFX complex, the X2BP polypeptide, and a NF-Y polypeptide are in solution.

The amount of complex formation in the presence of the test compound compared to the absence of the test compound. An increase or a decrease of complex formation in the presence of the test compound compared to the absence of the test compound indicates that the test compound is a modulator of MHC II gene expression. Optionally, one or more wash steps are performed prior to detection of the complex.

A compound that modulates MHC II gene expression is identified by bringing into contact a first polypeptide such as CIITA polypeptide, a RFXANK polypeptide, a RFXAP polypeptide or a RFX5 polypeptide, a second polypeptide such as a RFXANK polypeptide, a RFXAP polypeptide, or a RFX5 polypeptide and a test compound under conditions where the first polypeptide and the second polypeptide, in the absence of the compound, are capable of forming a complex; and determining the amount of complex formation. Optionally one or more wash steps are performed prior to detection of complex formation. An increase or decrease in the amount of complex formation in the presence of the test compound compared to the absence of the test compound indicates the compound modulates MHC II gene expression. Optionally, the first polypeptide is immobilized on a solid support and the second polypeptide further includes a detectable label.

Complex formation is detected by methods known in the art. For example, complex formation is detected by tagging one or more of the complex components, e.g., the MHC II promoter, the CIITA polypeptide or the RFX complex with a detectable label.

Detectable labels include horseradish peroxidase, alkaline phosphatase, glucose oxidase, peroxide, tetramethylbenzadine systems (tmb), biotin, digoxigenin, or fluorescein, allophycocyanin (APC), Texas red, Oregon green, dinitrophenol (DNP), lacZ, CAT, FITC, streptavidin, biotin, acceptor molecules, or donor molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. As used herein, the singular forms “a”, “and”, “or” and “the” are used to indicate plural references as well as singular, unless the context clearly dictates otherwise. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a schematic representation of two reporter gene constructs used in a cell-based reporter gene assay.

FIG. 2 is a flow chart of a cell-based reporter gene assay.

FIG. 3 is a schematic representation of the cascade of events leading to MHCII expression upon IFN-γ induction.

FIG. 4 is a schematic representation of an assay for detecting an interaction between CIITA and RFXANK.

FIG. 5 is a graph depicting colorimetric detection of the CIITA/RFXANK interaction in a 96 well format.

FIG. 6 is a graph showing the reproducibility of a colorimetric detection assay, wherein the signal to background ratio and Z value were S/B=11.78 and Z′=0.78, respectively.

FIG. 7 is a bar graph showing that the interaction between CIITA and RFXANK having an N-terminal Flag tag (Flag-RFXANK) is competed by purified RFXANK lacking the N-terminal Flag tag, such that equivalent amounts of Flag-RFXANK and RFXANK reduce the signal by approximately 50%.

FIG. 8 is a schematic representation of an assay for detecting an interaction between the RFX complex and a DR-promoter fragment interaction3.

FIG. 9 is a bar graph showing the specific binding of the RFX complex to a prototype MHC-II promoter (the DR-syn promoter), but not to a DR promoter in which the X box was mutated (the DR-X-mut promoter).

FIG. 10 is a schematic representation of an assay for detecting interactions between CIITA, the RFX complex and a promoter fragment.

FIG. 11 is a histogram depicting the detection of multiple interactions between CIITA, RFX and a promoter fragment and demonstrating that CIITA/RFX/DNA (i.e., promoter) interactions are dependent on RFX and CIITA.

FIG. 12 is a histogram depicting that RFX complex bound to CIITA discriminates between wild type and mutated MHC class II related promoters.

FIG. 13 is a schematic representation of a second assay for detecting interactions between CIITA, the RFX complex and a promoter fragment.

FIG. 14 is a histogram depicting that the RFX complex bound to CIITA discriminates between wild-type and mutated promoter fragments.

FIG. 15 is a schematic representation of an assay for detecting interactions between CIITA, the RFX complex and DNA in a solution-based format.

FIG. 16 is a histogram depicting detection of the CIITA/RFX:DNA interaction using a homogenous assay.

FIG. 17 is a schematic representation of a CIITA recruitment assay. Steps in the recruitment assay are shown, wherein (1) biotinylated fragments of the HLA-DRA gene promoter are immobilized in 96-well plates (X, X2, and Y, are RFX-, X2BP-, and NF-Y-binding sequence elements, respectively); (2) HRP-conjugated monoclonal antibody and extracts from human B cells are co-incubated in wells coated with promoter fragments. Binding of RFX, X2BP (CREB), NF-Y, CIITA, and the antibody is allowed to proceed; (3) Complexes formed at step (2) are washed several times; (4) the last wash is discarded and luminescence substrate is added; and (5) luminescence is read. The luminescence signal is proportional to the amount of CIITA associated with promoter fragments.

FIG. 18 is a histogram depicting the results of a CIITA recruitment experiment in which extract was incubated in the presence of either wild-type promoter fragments, or mutated promoter fragments which lack functional binding sites for RFX, X2BP (CREB), and NF-Y (binding sites inactivated through substitution mutagenesis).

### DETAILED DESCRIPTION OF THE INVENTION

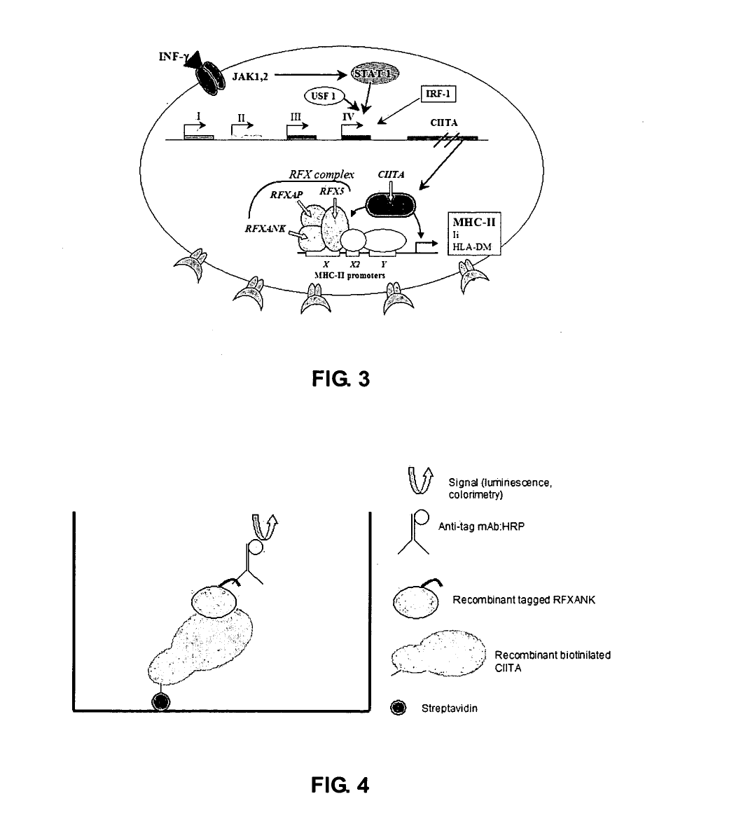

MHC class II expression depends on CIITA and RFX, two transcription factors that are highly selective for MHC class II genes. RFX is expressed ubiquitously, while CIITA expression is cell-specific and finely regulated. Hence, the pattern of MHC class II expression replicates faithfully the expression pattern of the gene encoding CIITA (MHC2TA). MHC2TA is expressed through a set of three cell-specific promoters, referred to as promoters I, III and IV. Promoters I and III are constitutively active in professional antigen-presenting cells, while in most other cell types CIITA expression is inducible with interferon gamma (IFNγ) through promoter IV. A schematic of the MHC class II promoter and interacting transcription factors is provided in FIG. 3.

The present invention provides methods for identifying (e.g., screening, detecting, characterizing, analyzing and quantifying) agents that modulate MHC-II promoter activity in cultured cells (referred to herein as a “cell-based reporter gene assay”). Test agents include, e.g., a small molecule compound, a protein, a protein fragment, a polypeptide, a peptide, an oligonucleotide such as an antisense molecule, an antibody or other ligand that is capable of selectively modulating MHC-II promoter activity.

In a cell-based assay, the phenotype of cultured cells is analyzed to identify agents that modulate expression of a first reporter gene operably linked to and controlled by an MHC-related promoter, e.g., a MHC class II promoter. These agents also modulate the expression of a second reporter gene that is operably linked to and driven by a non-MHC gene promoter (also referred to herein as an “unrelated promoter”), such that the agent differentially modulates the expression of the first reporter gene as compared to a second control reporter gene. By “differential modulation” is meant, for example, that the identified agent inhibits or reduces promoter expression and/or promoter activity of the MHC-related promoter to a greater degree than it inhibits or reduces promoter expression and/or promoter activity of the MHC-unrelated promoter. This inhibitory effect is specific to the MHC class II promoter. “Differential modulation” also signifies, for example, that the identified agent activates or induces promoter expression and/or promoter activity of the MHC-related promoter to a greater degree that it activates or induces promoter expression and/or promoter activity of the MHC-unrelated promoter.

Any reporter gene known to one skilled in the art is contemplated as being within the scope the invention. The reporter activities are similar (e.g., they are the same reporter activity), or the reporter activities are unrelated. For example, reporter constructs are available that can be assayed independently one from the other. A nonlimiting list of exemplary reporters includes genes encoding beta-galactosidase, chloramphenicol acetal transferase, luciferase (e.g., firefly luciferase, Renilla luciferase), RNAi or siRNA. Choice of the particular reporter, as well as the choice of MHC class II promoter and non-MHC class II gene promoter are within the skills of a person of ordinary skill in the art.

MHC-II-related promoters for use in cell-based assays described herein include, but are not limited to, a promoter for a gene encoding a protein in a HLA-DR complex, a promoter for a gene encoding a protein in a HLA-DP complex, a promoter for a gene encoding a protein in a HLA-DM complex and a promoter for a gene encoding a protein in a HLA-DQ complex. An exemplary promoter is a HLA-DRA or a HLA-DRB promoter. The promoter DNA is either wild-type promoter DNA, or mutated promoter DNA. Where mutated, the promoter sequence is modified as compared to the wild type through the use of substitution mutagenesis, or by any other method known to one skilled in the art.

In a cell-based assay, the first reporter gene construct and the second reporter gene construct are co-transfected into cells that express both the first and second reporter genes after IFN-γ induction. In a given assay, e.g., a screening assay, cells containing at least the first and the second reporter constructs are contacted with one or more test agents to be screened. A test agent has a modulatory effect on a reporter where the level of expression from the promoter constructs is different from the level of expression in a similar control cell population that has not been contacted with the test agent(s). An increase in reporter expression in the treated cell population compared to the control population indicates that the test agent is an activator of transcription. A decrease in reporter expression in the treated cell population compared to the corresponding control population indicates that the test agent is an inhibitor of transcription. A test agent is identified as a specific inhibitor of a MHC class II promoter where expression of a reporter under the control of an MHC class II promoter is decreased after being contacted with test agent, as compared to the level of expression in the same construct when the construct has not been contacted with the test agent. A test agent is also identified as a specific inhibitor of an MHC class II protein where expression of the reporter under the control of an MHC class II promoter is decreased relative to expression from a second independent (i.e., non-MHC class II) control reporter.

In addition to these cell-based assays, the present invention also provides methods for accurate and efficient detection of test agents that specifically modulate the interaction and/or activity of CIITA, RFXANK, RFXAP and RFX5 polypeptide factors. Test agents include, e.g., a small molecule compound, a protein, a protein fragment, a polypeptide, a peptide, an oligonucleotide such as an antisense molecule, an antibody or other ligand that selectively modulates the interaction and/or activity of CIITA, RFXANK, RFXAP and RFX5.

Assays are disclosed for identifying (e.g., screening, detecting, characterizing, analyzing and quantitating) the modulation of either protein:protein interactions, protein:DNA interactions, or both, wherein the interaction is involved in transcriptional regulation of MHC class II genes. These assays are referred to herein as “biochemical assays”. These biochemical assays detect the modulation, if any, in protein:protein or protein:DNA complex formation and/or interaction upon contact with a test agent. For example, the modulation is an inhibition of either one or more protein:protein and/or protein:DNA interactions such as the interaction between an MHC II promoter (or fragment thereof), one or more RFX complex proteins (e.g., RFXANK, RFXAP or RFX5 or fragments thereof), an X2BP polypeptide (or fragment thereof), an NF-Y polypeptide (or fragment thereof), a CIITA polypeptide (or fragment thereof) and combinations thereof. In a preferred embodiment, the inhibition is a specific inhibition of transcriptional regulation of MHC class II genes.

Protein:protein complexes to be assayed include, but are not limited to, one or more of: CIITA and RFXANK; CIITA and the RFX complex, wherein the RFX complex includes any one or more of RFXANK, RFXAP and RFX5; the RFX complex or any or more of its component factors with either X2BP (CREB), NF-Y, or both; and CIITA and the multi-protein platform formed by RFX complex, X2BP (CREB), and NF-Y. In a particular embodiment, the multi-protein platform formed by RFX complex, X2BP (CREB), and NF-Y is bound to promoter DNA.

The RFX complex and the X2BP (CREB) and NF-Y transcription factors are either in solution (including in a cell extract) or on a solid-state support. Any appropriate solid-state support known in the art is contemplated and within the scope of the invention. These proteins and/or complexes are optionally bound to promoter DNA. The MHC-II-related promoter DNA includes, but is not limited to, a promoter for a gene encoding a protein in a HLA-DR complex, a promoter for a gene encoding a protein in a HLA-DP complex, a promoter for a gene encoding a protein in a HLA-DM complex and a promoter for a gene encoding a protein in a HLA-DQ complex. For example, the promoter is a HLA-DRA or a HLA-DRB promoter. The promoter DNA is either a wild-type promoter, or a mutated promoter that lacks functional binding sites for its associated transcription factors, including but not limited to, e.g., RFX, X2BP (CREB), and NF-Y.

The disclosed individual polypeptides are either wild type or mutein, and are either provided in cell extracts or as recombinant proteins. A mutein polypeptide is defined as a polypeptide that differs by at least one amino acid residue that is not present in its corresponding wild type polypeptide. The at least one amino acid residue difference may be an addition, a deletion, a substitution, or any combination thereof of any one or more amino acids in the polypeptide as compared to the wild type. Suitable mutein polypeptides include those muteins having a modified function as compared to the wild type, or is altered in its interaction with other polypeptides or compounds. A complex of the one or more disclosed polypeptides contain all wild type polypeptides, all mutein variants, or a combination of wild type and mutein polypeptides.

The assays described herein are used to screen and/or identify compounds that modulate these interactions, wherein the modulation results in altered MHC class II gene activity. Compounds identified using these assays inhibit interactions between the various factors themselves or between these factors and DNA. For example, the inhibition of the various factor interactions specifically represses MHC class II expression.

The test agent is, for example, is a low molecular weight (LMW) compound, a medium molecular weight (MMW) compound, or a high molecular weight (HMW) compound. The compound modifies interaction between at least one of: (a) CIITA and RFXANK, (b) CIITA and the RFX complex, including any one or more of RFXANK, RFXAP and RFX5, (c) the RFX complex or its component factors with X2BP (CREB); (d) the RFX complex or its component factors with NF-Y; and (e) CIITA and the multi-protein DNA platform formed by RFX complex, X2BP (CREB), and NF-Y bound to promoter DNA. The promoter DNA includes a MHC class II promoter. The compounds modify the activity or interaction of the disclosed polypeptides, wherein the polypeptides are either wild type or muteins, and are provided, e.g., in cell extracts or as recombinant proteins.

The biochemical assays of the present invention are configured, for example, in a microwell assay format useful in moderate and high throughput screening applications. Test agents to be screened include bioactive agents such as one or more of a whole protein, a protein fragment, a polypeptide, a nucleotide, a peptidomimetic or a small molecules. The test agents are tested individually or provided in a library to be screened.

The biochemical assays of the present invention are used to study direct interactions between transcription factors CIITA, RFXANK, RFX5 and RFXAP. The methods are relevant to at least five classes of interactions. The first type of biochemical assays analyze direct interaction between any two of these proteins, such as, e.g., the interaction between CIITA and RFXANK. The second type of biochemical assays analyze the interaction of at least one protein with a protein complex, e.g., the interaction between CIITA and the RFX complex. The third type of biochemical assays analyze the direct interaction of two or more proteins in the presence of a DNA target sequence, e.g., when dealing with DNA-binding proteins or complexes. Exemplary target sequences are the MHC promoters and constructs described above. The fourth type of biochemical assays analyze the interactions of RFX complex with X2BP (CREB) and/or NF-Y in the context of promoter DNA. The fifth type of biochemical assays analyze the interactions of CIITA with the multi-protein-DNA platform formed by RFX complex, X2BP (CREB), and NF-Y bound to promoter DNA.

The biochemical assays of the present invention are used to study protein-protein interactions, as well as protein-DNA interactions. The biochemical assays of the present invention are also be used to study and validate the functional relevance of a particular protein-protein interaction, using mutated proteins in order to correlate loss of functional activity and loss of specific binding to a protein partner. Moreover, the biochemical assays of the present invention are used to screen, identify and characterize molecules (e.g., proteins, protein fragments, peptides, oligosaccharides, lipids, nucleic acids, or combinations thereof, or small molecules, synthesized compounds, compounds present in natural extracts, or libraries of one or more of the above) that affect the interactions between these proteins, e.g., interactions of CIITA, RFXANK, RFXAP and RFX5 polypeptide factors, or the RFX complex with X2BP (CREB), NF-Y, or both, with one another or with DNA.

Definitions:

The term “bioactive agent” or “bioactive molecule” refers to any therapeutic protein, peptide, polysaccharide, nucleic acid, lipid, carbohydrate, peptidomimetic, organic or inorganic compound, or other biologically active formulation for administration to a subject, such as a human or other mammal.

The term “compound library” refers to a mixture or collection of one or more compositions of matter generated or obtained in any manner. Preferably, the library contains more than one member or compositions of matter.

The term “fragment” or a “portion” of a polypeptide contains at least one fewer amino acid residues than the full-length polypeptide. The one or more deleted amino acids may be removed from the N-terminus, the C-terminus, or an internal portion. The term “fragment” or a “portion” of a nucleotide contains at least one fewer nucleic acid residue than the full-length polynucleotide. The one or more deleted nucleic acids may be removed from the 5′-end, the 3′end, or an internal portion of the given nucleotide fragment.

A “promoter sequence” is a generic term used throughout the specification to refer to one or more DNA sequences that induce or control transcription of DNA coding sequences with which they are operably linked. A “mutated promoter fragment” is a promoter having one or more residues altered from the nucleotide sequence of the wild-type promoter. The altered residue may be at least one residue making up a transcription factor binding site, a deletion or insertion of one or more promoter elements, or a silent change with no functional difference when assayed as compared to wild type.

In a variant embodiment with a mutant promoter containing an altered binding site, the ability of the corresponding transcription factor to bind the mutated site is modified as compared to binding of its wild type binding site.

The term “small molecule” refers to a compound having a molecular weight that is less than about 2500, less than about 2000, less than about 1500, less than about 1000, or less than about 750. An exemplary small molecule compound is a bioactive agent, for example a drug or pharmaceutically active moiety. Small molecules can be carbohydrates, lipids or other organic or inorganic molecules. A small molecule may be synthesized or may be a naturally occurring composition. A “synthetic organic compounds” refers to small molecule compounds generally having a molecular weight less than about 1500, preferably less than about 750, that are prepared by synthetic organic techniques, such as by combinatorial chemistry techniques.

The term “wild-type” refers to an allele of a gene or the protein product encoded by a gene that, when the allele is present in two copies in a subject, results in a wild-type phenotype. There can be several different wild-type alleles of a specific gene, since certain nucleotide changes in a gene may not affect the phenotype of a subject having two copies of the gene with the nucleotide changes.

I. Cell-based Assays

In the cell-based assays, the phenotype of cells transfected with at least one reporter gene are analyzed to identify agents that modify MHC class II expression. The cells are HTS-compatible to allow for rapid and efficient identification of such agents. Test agents that are inhibitors of MHC class II expression are identified by their ability to suppress expression of a first reporter gene operably linked to and controlled by a MHC class II-related gene promoter, e.g., the HLA-DRA promoter or the HLA-DRB promoter. Test agents that are activators of MHC class II expression are identified by their ability to induce expression of a first reporter gene operably linked to and controlled by a MHC class II-related gene promoter, such as the HLA-DRA promoter or the HLA-DRB promoter. The MHC class II related gene promoter is an IFNγ inducible promoter. Other MHC class II related gene promoters are known in the art and are included within the scope of this invention. A second reporter gene operably linked to and driven by an unrelated (i.e., non-MHC-II) gene promoter is provided as a control. For example, the second control reporter is operably linked to and driven by an IFN-γ inducible gene promoter, such that the first and second promoters are IFN-γ inducible promoters. Where the first and second are both IFN-γ inducible promoters, test agents are added, e.g., concomitantly, prior to or subsequent to IFN-γ induction.

The first reporter gene construct and the second reporter gene construct are co-transfected into cells. A wide variety of cells are used in the cell-based assays. For example, the cells are Me67.8 human melanoma cell lines.

Expression of the reporter in the first construct is compared to expression of the reporter in the second construct. The level of expression of the first reporter is independently assayable from the level of expression of the second reporter. The level of expression of the reports is detecting using a standard assay. The level of expression is detected, e.g., by assaying the concentration of transcription product (e.g., RNA) or the concentration of the transcribed and translated reporter gene product (e.g., polypeptide). Any RNA or polypeptide assay known to those skilled in the art is used to measure the level of expression of the reporters, and they are contemplated as being within the scope of the invention. Assays of the polypeptide product include, but are not limited to, e.g., physical detection of the polypeptide as well as detection of polypeptide activity. In certain embodiments, the assay includes detection of the activity of the transcribed and translated polypeptide expressed by the first, second, or both reporter genes. For example, at least one of the reporter genes is a luciferase gene. The luciferase is cloned, e.g., from the Renilla or firefly luciferase gene. In the cell based assay, the first reporter, for example, is a Renilla luciferase and the second reporter is a firefly luciferase.

II. Biochemical Assays

A. Solid-State Assays

In this type of assay one of the interacting partners (e.g., a protein, a protein complex or a DNA) is immobilized on a solid support. Exemplary solid supports include, but are not limited to, e.g., scaffolds such as a slide, well, tube or bead either by direct interaction with the solid support or via a capturing method. Any sold-state scaffold known to those skilled in the art is contemplated as part of this invention. Capturing methods include, but are not limited to, the use of antibodies directed against the protein or against tagged versions of the protein, the use of (strept)avidin-biotin interaction, metal chelating affinity, GST-glutathione affinity or other method used for protein or DNA immobilization by those skilled in the art.

In one embodiment, a second interacting partner is incubated with the immobilized partner. In given embodiments, the interaction is directly revealed if the second partner is labeled or where an indirect detection reagent is used, such as antibodies directed against the protein or protein tags, (strept)avidin or biotin based reagents, or other type of detection reagents used in the art. Detection methods include, but are not limited to, enzymatic activity, fluorescence, time-resolved fluorescence, polarized fluorescence, luminescence, radioactive labeling and scintillation proximity or other methods commonly used in the field of high throughput screening.

B. Homogeneous Assays

In this type of assay both partners (protein:protein complex or protein:DNA complex) interact in solution. The degree of interaction is detected by methods known to those skilled in the art, including, but not limited to, e.g., Fluorescence Resonance Energy Transfer (FRET and TR-FRET), Bioluminescence Resonance Energy Transfer (BRET), Scintillation, Proximity technology, and/or Fluorescence Polarization. In this format, each partner can be labeled directly or indirectly. Indirect labeling methods include, but are not limited to, using labeled antibodies directed against the protein or against tagged versions of the protein, using (strept)avidin-biotin interaction, metal chelating affinity reagent or GST-glutathione affinity reagents. Any direct or indirect labeling method known to those skilled in the art is contemplated as part of this invention.

III. Methods of Use

Agents identified using the cell-based and/or biochemical assays of the invention are useful in the treatment of diseases and disorders related to MHC class II expression. A broad review of MHC class II related diseases, and especially HLA-related diseases, are provided by the National Center for Biotechnology Information on its website entitled “Online Mendelian Inheritance in Man”, especially including, e.g., entries OMIM 142857 and OMIM 142860.

Disorders are know that are related to, or are a consequence of, an underlying mis-regulation of molecules and proteins encoded by one or more genes of the MHC class II family. Specific diseases are, e.g., due to increased gene expression, decreased gene expression, gene expression in tissues where the gene is not normally expressed, or to an adverse interaction between an expressed MHC class II-related molecule and one or more other biological molecules or systems. Mis-regulated expression includes, but is not limited to, an inappropriate or aberrant temporal expression, an inappropriate or aberrant spatial expression, an inappropriate or aberrant level of expression, chronic expression, and the like. For example, the adverse interaction is a reaction by the immune system, such as an autoimmune reaction.

Agents identified using the assays of the invention are therefore useful in modulating MHC class II expression in a patient in need thereof. Agents identified as specifically upregulating MHC class II expression as useful in treating diseases related to decreased expression of a MHC class II gene, including but not limited to, e.g., Bare Lymphocyte syndrome. Agents identified as specifically inhibiting MHC class II expression as useful in treating diseases related to chronic, aberrant and/or inappropriate expression of a MHC class II gene, including but not limited to, e.g., IDDM.

Agents identified using the assays of the invention are used prophylactically, to alleviate a symptom associated with an autoimmune disease, an inflammatory disorder and/or a transplant-related disorder, or to treat a subject in need of treatment who has, or is at risk of having, an autoimmune disease, an inflammatory disorder and/or a transplant-related disorder.

A. Autoimmune Diseases

Agents identified using the assays of the invention are sued to treat or alleviate a symptom associated with autoimmune diseases, such as, for example, connective tissue-related diseases, neuromuscular-related diseases, endocrine-related diseases, gastrointestinal-related diseases, and autoimmune-related skin diseases. Connective Tissue Diseases include, but are not limited to, e.g., Systemic Lupus Erythematosus (SLE); Rheumatoid Arthritis; Systemic Sclerosis (Scleroderma); and/or Sjögren's Syndrome. Neuromuscular Diseases include, but are not limited to, e.g., Multiple Sclerosis (MS); Myasthenis Gravis; and/or Guillain-Barré Syndrome. Endocrine Diseases include, but are not limited to, e.g., Hashimoto's Thyroiditis; Graves' disease; and/or Insulin-Dependent (Type 1) Diabetes. Gastrointestinal Diseases include, but are not limited to, e.g., Inflammatory Bowel Disease such as, e.g., Crohn's Disease and/or Ulcerative Colitis. Autoimmune Skin Diseases include, but are not limited to, e.g., Psoriasis and/or Psoriatic Arthritis. Other Autoimmune Diseases include, but are not limited to, e.g., Vasculitis Syndromes; Hematologic Autoimmune Diseases; and/or Uveitis.

Other autoimmune-related conditions and diseases include, but are not limited to, e.g., Acquired Immunodeficiency Syndrome (AIDS; a viral disease with an autoimmune component); Alopecia Areata; Ankylosing Spondylitis. Antiphospholipid Syndrome; Autoimmune Addison's Disease; Autoimmune Hemolytic Anemia; Autoimmune Hepatitis; Autoimmune Inner Ear Disease (AIED); Autoimmune Lymphoproliferative Syndrome (ALPS); Autoimmune Thrombocytopenic Purpura (ATP); Behcet's Disease; Cardiomyopathy; Celiac Sprue-Dermatitis Hepetiformis; Chronic Fatigue Immune Dysfunction Syndrome (CFIDS); Chronic Inflammatory Demyelinating Polyneuropathy (CIPD); Cicatricial Pemphigold; Cold Agglutinin Disease; Crest Syndrome (see also Raynaud's phenomena and Scleroderma); Crohn's Disease; Degos' Disease; Dermatomyositis-Juvenile; Discoid Lupus; Essential Mixed Cryoglobulinemia; Fibromyalgia-Fibromyositis; Graves' Disease; Guillain-Barré Syndrome; Hashimoto's Thyroiditis; Idiopathic Pulmonary Fibrosis; Idiopathic Thrombocytopenia Purpura (ITP); IgA Nephropathy; Insulin dependent diabetes mellitus; Juvenile Chronic Arthritis (Still's Disease); Juvenile Rheumatoid Arthritis; Ménière's Disease; Mixed Connective Tissue Disease; Multiple Sclerosis; Myasthenia Gravis; Pernacious Anemia; Polyarteritis Nodosa; Polychondritis; Polyglandular Syndromes; Polymyalgia Rheumatica; Polymyositis and Dermatomyositis; Primary Agammaglobulinemia; Primary Biliary Cirrhosis; Psoriasis; Psoriatic Arthritis; Raynaud's Phenomena; Reiter's Syndrome; Rheumatic Fever; Rheumatoid Arthritis; Sarcoidosis; Scleroderma (Progressive systemic sclerosis (PSS), also known as systemic sclerosis (SS)); Sjögren's Syndrome; Stiff-Man Syndrome; Systemic Lupus Erythematosus; Takayasu Arteritis; Temporal Arteritis/Giant Cell Arteritis; Ulcerative Colitis; Uveitis; Vitiligo; and Wegener's Granulomatosis.

B. Inflammation

Agents identified using the assays of the invention are used to treat or alleviate a symptom associated with inflammation-related diseases such as, for example, Alzheimer's disease; Asthma; Atopic allergy; Allergy; Atherosclerosis; Bronchial Asthma; Eczema; Glomerulonephritis; Graft vs. host disease; Hemolytic anemias; Osteoarthritis; Sepsis; Stroke; Transplantation of tissue and organs; Vasculitis; and Ventilator induced lung injury.

C. Transplantation

Agents identified using the assays of the invention are also used as immunosuppression agents to treat a patient prior to, during and/or after organ or tissue transplantation (e.g., skin transplant, kidney transplant, pancreatic islet transplant, cardiac transplant). These immunosuppressive compositions include a pharmaceutically acceptable carrier and a therapeutically active composition identified through a method of the invention, or pharmaceutically acceptable salts or hydrates thereof.

IV. Combination Therapies

In one embodiment, the agents identified using the assays of the invention are formulated as compositions used to treat an immune-related disorder. These compositions are administered in combination with any of a variety of known anti-inflammatory and/or immunosuppressive compounds. Suitable anti-inflammatory and/or immunosuppressive compounds for use with the compounds of the invention include, but are not limited to, methotrexate, cyclosporin A (including, for example, cyclosporin microemulsion), tacrolimus, corticosteroids, statins, interferon beta, nonsteroidal anti-inflammatory drugs (NSAIDs) and the disease-modifying anti-rheumatic drugs (DMARDs).

The compositions of the invention are used as immunosuppression agents in organ or tissue transplantation. As used herein, “immunosuppression agent” refers to an agent whose action on the immune system leads to the immediate or delayed reduction of the activity of at least one pathway involved in an immune response, whether this response is naturally occurring or artificially triggered, whether this response takes place as part of the innate immune system, the adaptive immune system, or both. These immunosuppressive compositions are administered to a subject prior to, during and/or after organ or tissue transplantation. For example, the compositions of the invention are used to treat or prevent rejection after organ or tissue transplantation.

In one embodiment, the compositions used to treat an immune-related disorder are administered in combination with any of a variety of known anti-inflammatory and/or immunosuppressive compounds. Suitable known compounds include, but are not limited to methotrexate, cyclosporin A (including, for example, cyclosporin microemulsion), tacrolimus, corticosteroids, statins, interferon beta, Remicade (Infliximab), Enbrel (Etanercept) and Humira (Adalimumab).

For example, in the treatment of rheumatoid arthritis, the compositions of the invention can be co-administered with corticosteroids, methotrexate, cyclosporin A, statins, Remicade (Infliximab), Enbrel (Etanercept) and/or Humira (Adalimumab).

In the treatment of uveitis, the compositions can be administered in conjunction with, e.g., corticosteroids, methotrexate, cyclosporin A, cyclophosphamide and/or statins. Likewise, patients afflicted with a disease such as Crohn's Disease or psoriasis can be treated with a combination of a composition of the invention and Remicaid (Infliximab), and/or Humira (Adalimumab).

Patients with multiple sclerosis can receive a combination of a composition of the invention in combination with, e.g., glatiramer acetate (Copaxone), interferon beta-1a (Avonex), interferon beta-1a (Rebif), interferon beta-1b (Betaseron or Betaferon), mitoxantrone (Novantrone), dexamethasone (Decadron), methylprednisolone (Depo-Medrol), and/or prednisone (Deltasone) and/or statins.

Inhibitory compounds are also useful in treating GVHD and other unwanted immune responses. Prophylactic treatments are also contemplated within the scope of the invention. The particular pharmaceutical formulations, therapeutically effective amount and treatment regimen for a patient in need of such treatment are within the skill of a practitioner in the art.

The invention is further described in the following examples, which do not limit the scope of the invention described in the claims.

### EXAMPLE 1

Cell Based Assay

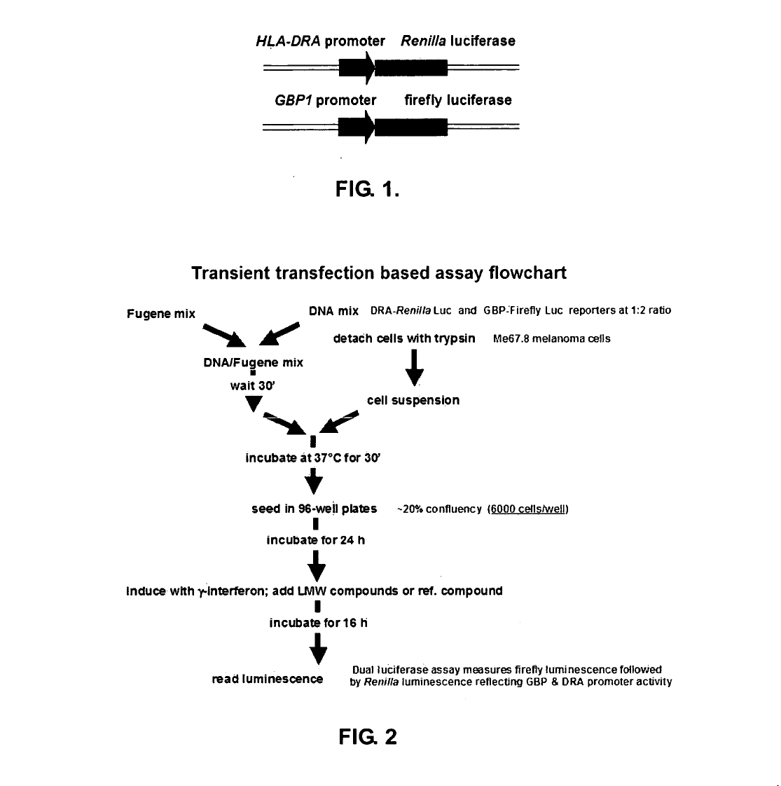

Reporter gene assay: A phenotype-based, high through-put screen (HTS)-compatible cell-based assay for the identification of inhibitors of MHC class II expression was developed. A reporter gene (Renilla luciferase) controlled by an MHC class II promoter (HLA-DRA), and a control reporter (firefly luciferase) driven by an IFN-γ-inducible gene promoter other than an MHC class II promoter (FIG. 1) are co-transfected into cells that express these two genes after IFN-γ induction (e.g., Me67.8 human melanoma cell line). It is recognized that IFN-γ induces MHC class II promoters. A test compound is added into the cell culture, e.g., concomitantly, prior to or subsequent to IFN-γ induction. After a time lag of one to several hours (e.g., 16 hours) a standard Dual Luciferase Reporter assay (DLR) is performed. Compounds that inhibit the induction of the Renilla luciferase, but not the firefly luciferase, are identified and characterized as selective MHC class II inhibitors. A flow chart of the assay is presented in FIG. 2.

### EXAMPLE 2

Assay for CIITA-RFXANK Interaction

Assays have been developed to directly determine interactions between CIITA and RFXANK. A graphic depiction of the assay is provided in FIG. 4. This assay is suitable for determination of interactions between CIITA and any member of the RFX complex, such as RFX5 and RFXAP.

Plasmids. The CIITA gene sequence was cloned into a modified pBAC-2 cp vector (Novagen) with a biotinylation tag inserted at the 5′ end of the gene. The RFXANK gene sequence containing a Flag tag at its N-terminus was cloned into a pFBHT vector (Invitrogen). These plasmids were used to generate recombinant baculoviruses according to standard procedures. Protein production: Sf9 cells grown in SF-900II medium (Invitrogen) were infected at MOI of 10 for 72 h before harvesting. To obtain in vivo biotinylated CIITA, Sf9 cells were co-infected with an E. coli biotin ligase expressing baculovirus (BirA gene) at a MOI of 1. Protein were purified using metal chelating affinity chromatography on HiTrap chelating column (Pharmacia). Purified fractions were pooled and quantified using ESL protein quantification kit (Roche) and the extent of CIITA biotinylation was estimated by pulldown experiments with Streptavidin-coated magnetic beads followed by PAGE analysis. These experiments showed that biotinylation level was close to 100%.

Assay. Biotinylated-CIITA (0.5 μg) and 0.8 μg of Flag-RFXANK in 100 μl of assay buffer (50 mM Hepes pH7.8; 10% glycerol; 5 mM MgCl₂; 0.1 mM EDTA; 0.01% NP-40; 200 mM KCl; 0.5% BSA; 0.1% Tween 20; 2 mM DTT) were incubated in a streptavidin-coated microwell for 2 h at room temperature. Wells containing no protein, only biotinylated CIITA or Flag RFXANK were included as controls. Wells were washed 5 times with 200 μl of wash solution (PBS; 0.05% Tween 20). 100 μl of Assay Buffer containing 0.5 μg/ml M2:HRP antibody (Sigma) was added to each well and incubated for 1 h at room temperature. Wells were washed 5 times with 200 μl of wash solution (PBS; 0.05% Tween 20) and the adequate substrates for HRP were added to the well for either colorimetry, fluorescence or luminescence detection.

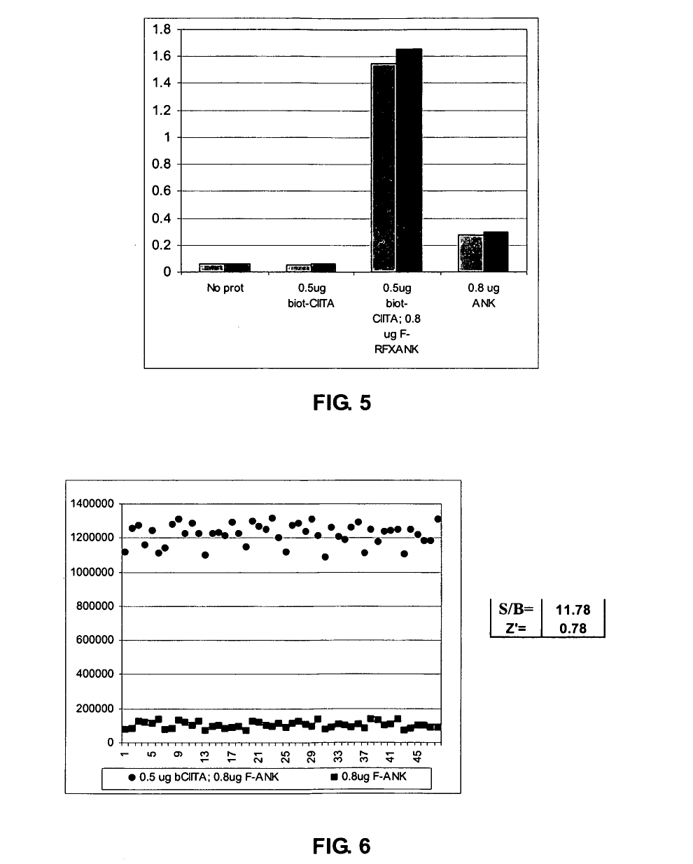

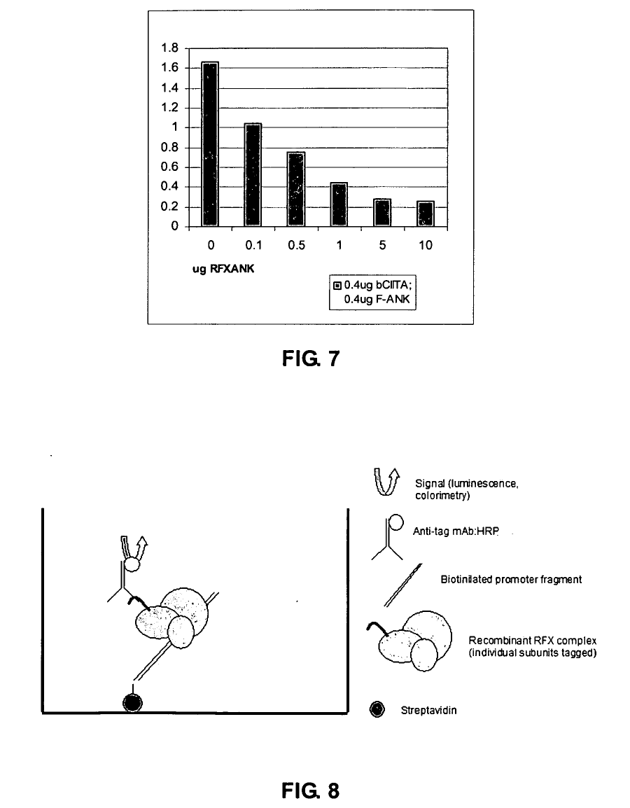

Results for such an assay using a colorimetric detection are shown in FIG. 5. To examine its reproducibility, the assay and negative control were performed 48 times. Experimental results are provided in FIG. 6. The values obtained give a signal to background ratio over 11 and a Z value of 0.78 for that assay, thus indicating that it very reproducible and very well suited for HTS (Zhang, Chung and Oldenburg, 1999. J Biomol Screen. 1999;4(2):67-73.) To test the specificity of the assay, competition experiments were performed with purified RFXANK lacking the Flag tag at its N-terminus. Increasing amounts of RFXANK were added to the wells containing biotinylated CIITA and Flag-RFXANK in the same assay as described above. This experiment shows that Flag-RFXANK is specifically competed by RFXANK for binding to CIITA in a dose-dependent manner. Experimental results are provided in FIG. 7.

Using similar methods, various embodiments of the present invention provide for the analysis of the following additional interactions: RFXAP-CIITA; RFX5-CIITA; RFX5-RFX5, RFXANK-RFXAP.

### EXAMPLE 3

Assay for RFX Complex/DR-Promoter Interaction

Assays have been developed to directly determine interactions between the RFX complex and an MHC class II promoter. A graphic depiction of an exemplary assay is provided in FIG. 8.

Plasmids. The RFXANK gene sequence containing a Flag tag at its N-terminus and the RFXAP gene sequence containing an HA tag at its N-terminus were cloned into the pFastBac Dual vector (Invitrogen) that allows for the co expression of two gene in insect cells. The RFX5 coding sequence was cloned into the pFBHT vector (Invitrogen). These plasmids were used to generate recombinant baculoviruses according to standard procedures. Protein production: Sf9 cells grown in SF-900II medium (Invitrogen) were co infected at MOI of 5 for each virus for 48 h before harvesting. Cells were lysed in lysis buffer (50 mM Tris-Cl pH8.5; 5 mM β-Me; 200 mM KCl; 1% NP-40) supplemented with Complete (EDTA-free) protease inhibitor cocktail (Roche). The lysed extracts were centrifuged at 10,000 g for 30 minutes at 4° C. Glycerol was added to the supernatant to 50% final concentration and stored at −20° C.

MHC-II promoter and control promoter. Biotinylated DR-Syn promoter (a prototype MHC-II promoter, Tsang et al., 1990) and DR-X-mut (the same promoter in which the X box was mutated) were obtained by PCR amplification performed on plasmids containing these respective promoters. The RFX complex is unable to bind to the mutated X box sequence (Hasegawa et al., 1991). The following oligonucleotides were used for PCR amplification: Biot-AAGCTTGTATCTTGTGTCCT-3′ (SEQ ID NO:1) and 5′-GGTGTAATAGAGTCTGACCA-3′ (SEQ ID NO:2). However, one of ordinary skill in the art will recognize that the present invention provides any fragment of an MHC-II promoter that is capable of binding to the RFX complex, or a component thereof.

Assay. Biotinylated DR-Syn or DR-X-mut promoters (2.5 pmole) were added to streptavidin-coated microwells in suspension buffer (PBS; 1% BSA) and incubated for 1 h at 4° C. Wells were washed 5 times with 200 μl of wash solution (PBS; 0.05% Tween 20). Assay Buffer (100 μl 50 mM Hepes pH7.8; 10% glycerol; 5 mM MgCl₂; 0.1 mM EDTA; 0.01% NP-40; 200 mM KCl; 1% BSA; 0.1% Tween 20; 2 mM DTT) containing 5 μl of RFX extract and 5 μg of E. coli DNA (as non-specific competitor) were added to the wells and incubated for 2 h at 4° C. Wells were washed 5 times with 200 μl of wash solution (PBS; 0.05% Tween 20). Assay Buffer (100 μl) containing 0.5 μg/ml Ab anti-HA:HRP antibody (Roche) was added to each well and incubated at room temperature for 1 h. Wells were washed 5 times with 200 μl of wash solution (PBS; 0.05% Tween 20) and colorimetric detection was used as readout.

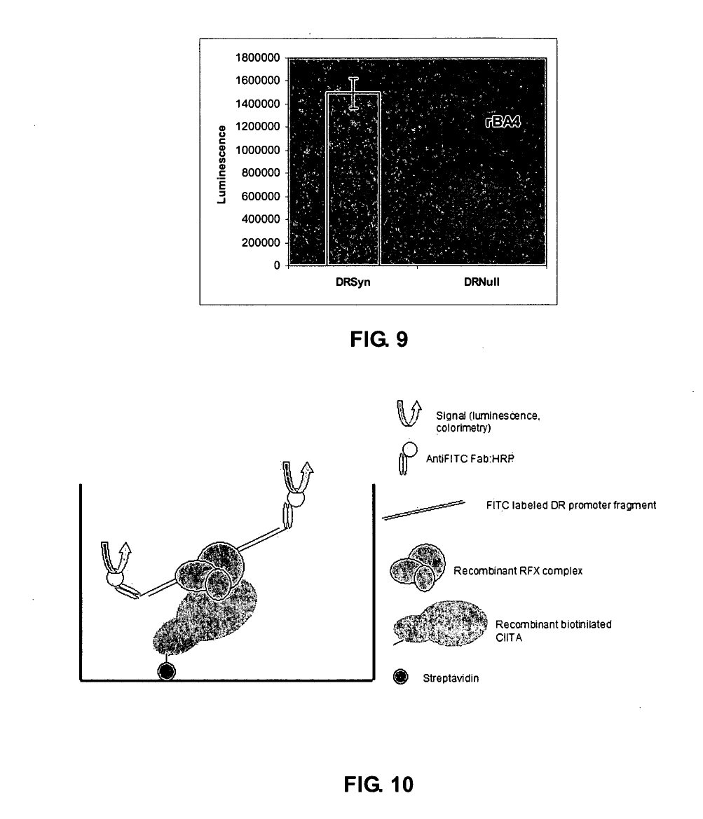

The anti-HA antibody allowed for the detection of the RFXAP subunit of the RFX complex and therefore the assay monitored the presence of that protein. However, the binding of the complex to the promoter X box is mediated by the DNA-binding RFX5 subunit. The results shown in FIG. 9 indicate that the RFX complex co-expressed in insect cells binds specifically to the DR-syn promoter and not to the DR-X-mut promoter. Thus this assay is suitable for the screening of molecules that inhibit the binding of the RFX complex to the MHC-II promoter and/or molecules inhibiting RFX complex assembly. This assay is also suitable for the screening of molecules that bind to the MHC-II promoter and competitively inhibit the binding of the RFX complex to the MHC-II promoter.

### EXAMPLE 4

Assay for CIITA-RFX Complex/DR-Promoter Interaction

Assays have been developed to directly determine interactions between CIITA and the RFX complex/DR-promoter. A graphic depiction of the assay is provided in FIG. 10.

Plasmids. The RFXANK gene sequence containing a Flag (F-) tag at its N-terminus, the RFXAP gene sequence containing an haemagluttinin (HA-) tag at its N-terminus as well as the RFX5 coding sequence were cloned into the pFBHT vector (Invitrogen). Insect cell culture, virus generation and protein production was performed as described above. Each RFX subunit was purified individually using metal chelating affinity chromatography on HiTrap chelating column (Pharmacia). Purified fractions were pooled and quantified using ESL protein quantification kit (Roche). RFX complex was reconstituted in vitro by mixing stoichiometric amounts of each RFX subunit. Recombinant biotinylated-CIITA was provided in the form of extracts from Sf9 cells infected with the baculovirus described in Example 2. This shows that recombinant CIITA both from extracts and in a purified form can be used in such assays. FITC-labeled DR-promoter probes: wild-type (DRsyn) and mutant promoter fragments (DRnull) were obtained by annealing the following oligonucleotides: 5DRsynFITC 5′-TATCTTGTGT CCTGGACCCT TTGCAAGGGC CCTTCCCCTA GCAACAGAT GCGTCATCTC GAGATATTTT TCTGA-3′ (SEQ ID NO:3); 3DRsynFITC 5′-TCAGAAAAAT ATCTCGAGAT GACG CATCTG TTGCTAGGGG AAGGGCCCTT GCAAAGGGTC CAGGACACAA GATA-3′ (SEQ ID NO:4); 5DRnullFITC 5′-TATCTTGTGT CCTTACATAG CGTACGTGGC CCAGAGATAC TATG CAGAGA CAAGTTCTCG AGATATTTTT GTGA-3′ (SEQ ID NO:5); 3DRnulIFITC 5′-TCACAAAAAT ATCTCG AGAA CTTGTCTCTG CATAGTATCT CTGGGCCACG TACGCTATGT AAGGACACAA GATA-3′ (SEQ ID NO:6).

Assay. 1 μl of Sf9 cell extract containing approximately 1 pmol of biotinylated-CIITA, 1 pmol of recombinant RFX complex, 1 pmol of DR promoter fragment, 0.5 μl of Fab anti-FITC:HRP (Roche) and 5 μg of sonicated E. coli DNA (double stranded) were mixed in 100 μl of assay buffer (50 mM Hepes pH7.8; 10% glycerol; 5 mM MgCl₂; 0.1 mM EDTA; 0.01% NP-40; 200 mM KCl; 0.5% BSA; 0.1% Tween 20; 2 mM DTT). This mix was then added to a streptavidin-coated microwell and incubated for 2 hrs at 4° C. The wells were then washed five times with assay buffer and the adequate substrates for horse radish peroxidase (HRP) was added to the well for either colorimetry, fluorescence or luminescence detection. In this assay CIITA is immobilized via a streptavidin-biotin interaction and allows for the sequential binding of the RFX complex and the DR promoter fragment. This DNA fragment can then be detected directly by measuring FITC fluorescence or using anti-FITC Fab fragments coupled to HRP for signal amplification. A schematic of this assay is provided in FIG. 10.

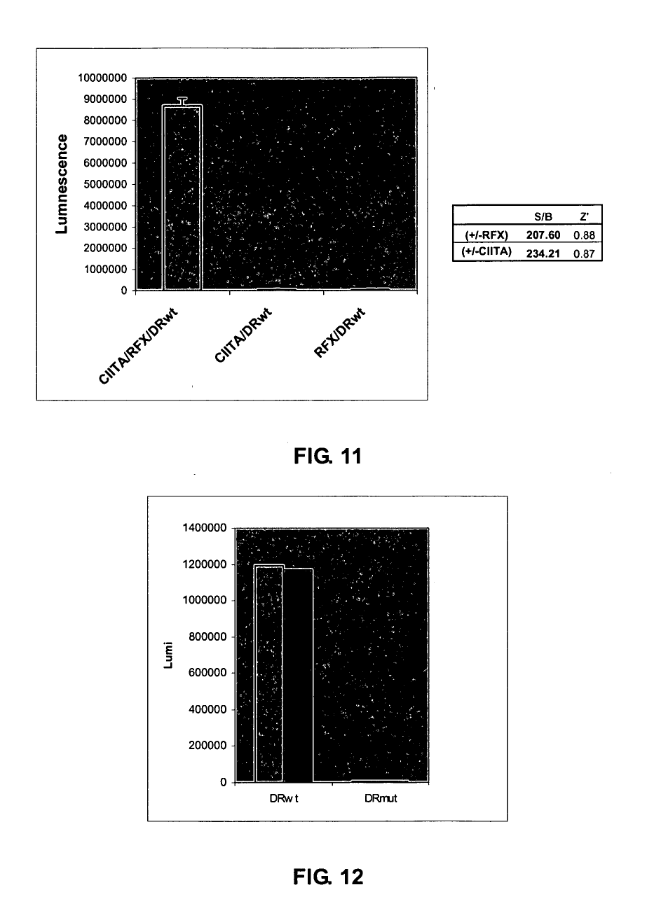

The results in FIG. 11 show that the multiple interactions between CIITA, RFX and DNA can be detected in this assay and that the signal is dependent on the presence of both CIITA and the DNA-binding RFX complex. The signal to background ratios and Z′ factors obtained indicate that this assay is suitable for screening of compounds that modulate IFN gene expression, such as high throughput screening methods. Furthermore, the RFX complex is unable to bind to mutated DR promoter, indicating that in this context RFX maintains its specificity for its cognate DNA sequence. Experimental results are shown in FIG. 12. Thus this assay is useful for promoter and protein mutagenesis studies as well as the identification of any compounds, including small molecules, peptides, antibodies and other biomolecules that alter CIITA-RFX interaction, RFX complex formation or stability, and RFX-DNA interaction.

### EXAMPLE 5

Assay for CIITA-RFX Complex/DR-Promoter Interaction

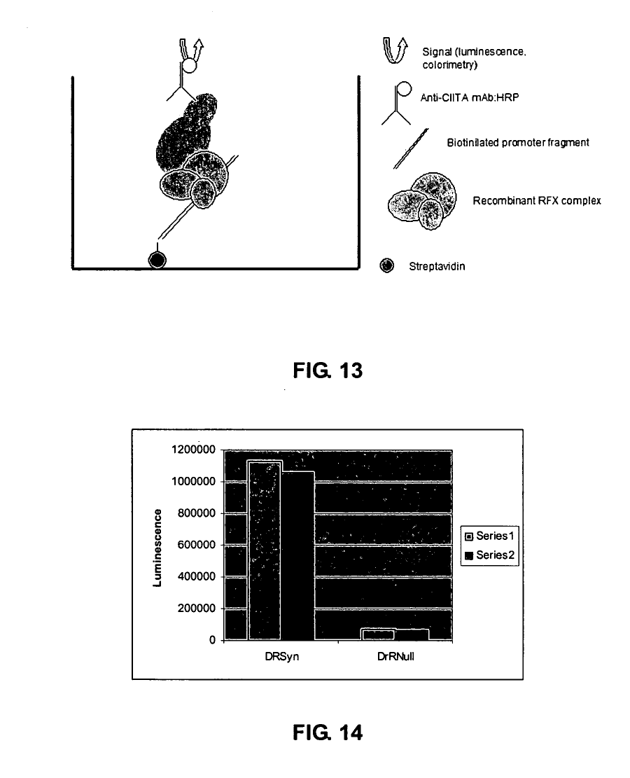

Assays have been developed to directly determine interactions between CIITA and the RFX complex/DR-promoter. A graphic depiction of the assay is provided in FIG. 13 and typical results are provided in FIG. 14. This solid phase assay is similar to that provided in Example 4, but is performed in an opposite orientation by immobilizing the promoter fragments to the well, and detecting CIITA that is recruited to the DNA via interaction with RFX.

### EXAMPLE 6

Assay for CIITA-RFX Complex/DR-Promoter Interaction.

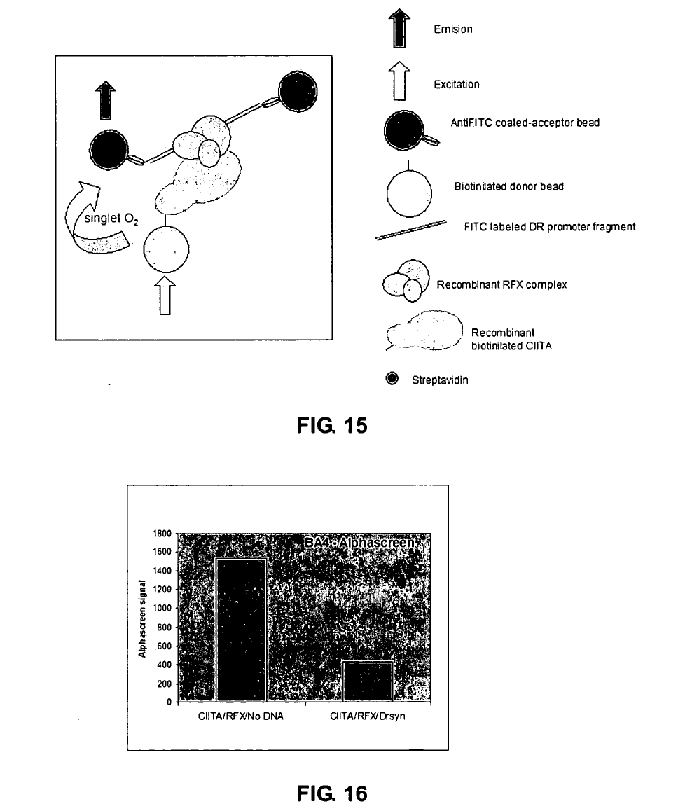

Assays have been developed to directly determine interactions between CIITA and the RFX complex/DR-promoter. This assay is a variant of the assay described in Example 4. The interaction between CIITA, RFX and DNA can be assayed in a solution format, such as Alpha Screen technology (Perkin Elmer). A depiction of the assay and typical results are shown in FIGS. 15 and 16, respectively. In this assay, CIITA is operably linked to a donor bead and the DR promoter fragment is operably linked to an acceptor bead. Generally, the donor and acceptor beads are coated with a material such as hydrogel that provides functional groups for bioconjugation. The donor bead contains a photosensitizer that converts ambient oxygen into a more excited singlet oxygen state. The acceptor bead contains a chemiluminescer and a fluorophore. When CIITA comes into proximity with the DR promoter fragment, the excited singlet oxygen produced by the donor bead contacts the acceptor bead and activates the chemiluminescer, which further activates the fluorophore, which subsequently emit light at 520-620 nm. The donor bead can be conjugated to CIITA by any means known in the art, such as streptavidin-biotin. The acceptor bead can be conjugated to the DR promoter by any means known in the art, such as when the promoter is labeled with FITC and the acceptor bead is coated with an anti-FITC antibody.

### EXAMPLE 7

Assay for Interactions Between CIITA and the Multi-Protein-DNA Platform Formed by RFX Complex, X2BP (CREB), and NF-Y Bound to Promoter DNA

The CIITA recruitment assay is an improvement over pull-down assays known in the art. (See, Masternak et al., 2000. Genes & Development 14: 1156-1166.) One advantage of the present invention is the ability to use the methods disclosed herein in high-throughput screening applications. In one embodiment, immobilized MHCII promoter fragments are incubated with Whole Cell Extracts (WCE) from human B cells containing CIITA, RFX, X2BP (CREB), NF-Y, and other factors. RFX, X2BP (CREB), and NF-Y bind promoter DNA and recruit CIITA. After washes, CIITA associated with an immobilized promoter fragment is detected with a specific reagent (e.g., a HRP-conjugated anti-CIITA antibody). Since CIITA recruitment depends on prior binding of RFX, X2BP (CREB), and NF-Y to promoter DNA, the CIITA recruitment assay detects inhibitory compounds that interfere at these steps.

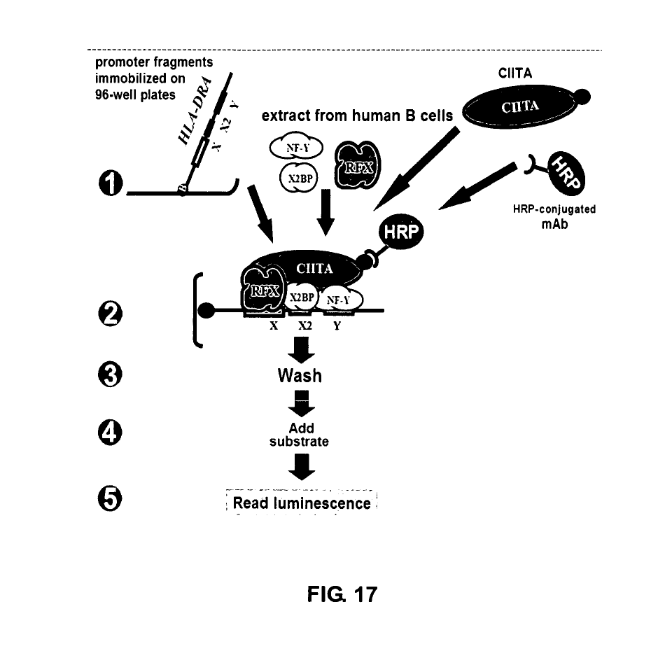

A representation of the CIITA recruitment assay is illustrated in FIG. 17. As shown in Part 1 of the schematic in FIG. 17, biotinylated fragments of the HLA-DRA gene promoter are immobilized in 96-well plates. X, X2, and Y, represent the RFX-, X2BP-, and NF-Y-binding sequence elements, respectively. In Part 2, extracts from human B cells and HRP-conjugated monoclonal antibody are co-incubated in wells coated with promoter fragments. Binding of RFX, X2BP, NF-Y, CIITA, and the antibody is allowed to proceed. In Part 3, complexes formed at step (2) are washed one or more times. In Part 4, the last wash is discarded and luminescence substrate is added. In Part 5, luminescence is read. The luminescence signal is proportional to the amount of CIITA associated with promoter fragments. The experimental details of an exemplary version of the assay are specified below.

Reagents: A list of reagents and buffers used are as follows: HLA-DRA gene promoter fragments (136 bp in length, spanning a region from −155 to −20 with respect to the start of transcription) biotinylated at the ORF-distal end (i.e., the “−155 bp” end); Streptawell white 96-well plates (Roche); Whole Cell Extracts (WCE) from RJ6.4 B cell line (Masternak et al., 2000) expressing HA-tagged CIITA; Non-specific competitor DNA: single-stranded E. Coli DNA, double-stranded polydldC•epolydldC; Extract Dilution Buffer: 20 mM Hepes pH7.9; 20% glycerol; 0.1 mM EDTA; 0.01% NP-40; 9 mM MgCl2; 1 mM DTT; Complete™ cocktail of protease inhibitors (Roche); 5 μg/mL leupeptin; 1 mM PMSF; 0.5 mM NaF; 0.5 mM Na₃VO₄; Wash Buffer: 20 mM Hepes pH7.9; 20% glycerol; 0.1 mM EDTA; 0.01% NP-40; 6 mM MgCl2; 100 mM KCl; 1 mM DTT; 1 mg/mL BSA; Rat anti-HA HRP-conjugated “high affinity” monoclonal antibody (Roche); and Detection Reagent: Supersignal ELISA Pico Chemiluminescent Substrate (Pierce).

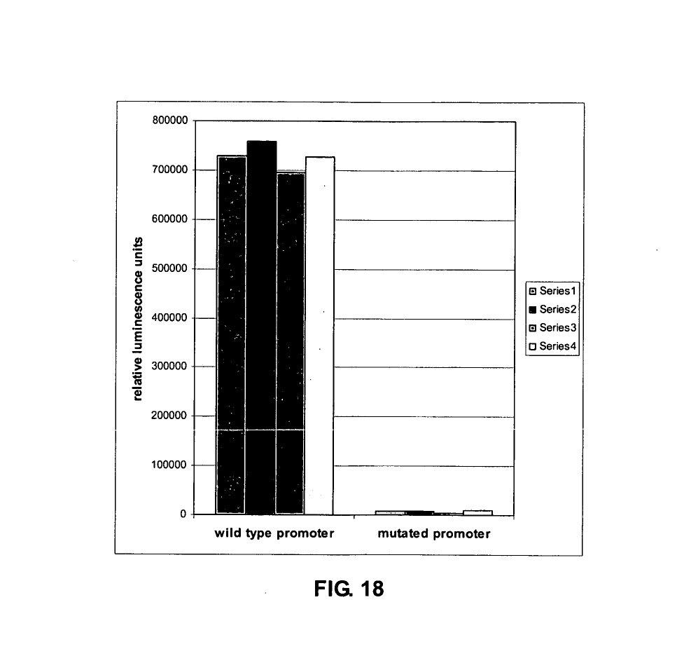

Assay: In this embodiment of the assay, promoter fragments (0.3 pmole per well) are immobilized in 96-well plates and incubated with extracts from RJ6.4 cells (8 μl extract per well, diluted 2× with Extract Dilution Buffer) in the presence of competitor DNA (150 ng/μl each) and HRP-conjugated anti-HA antibody for two hours at RT. After washing the wells 3× with Wash Buffer, Detection Reagent is added and luminescence signals are measured in a microplate reader. An example of results obtained with this assay is shown in FIG. 18.

### REFERENCES CITED

Hasegawa S L, Sloan J H, Reith W, Mach B, Boss J M. Regulatory factor-X binding to mutant HLA-DRA promoter sequences. Nucleic Acids Res. 1991 Mar. 25;19(6): 1243-9.
Masternak, K., A. Muhlethaler-Mottet, J. Villard, M. Zufferey, V. Steimle, W. Reith. CIITA is a transcriptional coactivator that is recruited to MHC class II promoters by multiple synergistic interactions with an enhanceosome complex. Genes Dev. 2000;14:1156-66.
Reith W, Mach B. The bare lymphocyte syndrome and the regulation of MHC expression. Annu Rev Immunol. 2001;19:331-73.
Tsang S Y, Nakanishi M, Peterlin B M. B-cell-specific and interferon-gamma-inducible regulation of the HLA-DR alpha gene. Proc Natl Acad Sci USA. 1988 November;85(22):8598-602.
Zhang J H, Chung T D, Oldenburg K R. A Simple Statistical Parameter for Use in Evaluation and Validation of High Throughput Screening Assays. J Biomol Screen. 1999;4(2):67-73

### Equivalents

Those skilled in the art will recognize, or be able to ascertain using no more than routine experimentation, numerous equivalents to the specific procedures described herein. Such equivalents are considered to be within the scope of the present invention. Various substitutions, alterations, and modifications may be made to the invention without departing from the spirit and scope of the invention. Other aspects, advantages, and modifications are within the scope of the invention. The contents of all references, issued patents, and published patent applications cited throughout this application are hereby incorporated by reference. The appropriate components, processes, and methods of those patents, applications and other documents may be selected for the present invention and embodiments thereof. Other aspects, advantages, and modifications are considered to be within the scope of the following claims.

1. A method of identifying a compound that modulates MHC II gene expression comprising:
a. contacting a test cell with a test compound wherein said cell comprises a nucleic acid comprising a reporter gene operably linked to an MHC II promoter or fragment thereof; and
b. comparing the amount of reporter gene expression in said test cell to a control cell that has not been contacted with said test compound,
wherein an increase or a decrease of said reporter gene expression in said test cell compared to said control cell indicates that said test compound is a modulator of MHC II gene expression.

2. The method of claim 1, said reporter gene encodes a protein that produces a detectable signal.

3. The method of claim 1, wherein said reporter gene encodes for a bioluminescent protein.

4. The method of claim 3, wherein said bioluminescent protein is ferredoxin IV, green fluorescent protein, red fluorescent protein, cyan fluorescent protein, yellow fluorescent protein, blue fluorescent protein, the luciferase family and the aequorin family, the phycobiliproteins (including B-Phycoerythrin (B-PE), R-Phycoerythrin (R-PE), and Allophycocyanin (APC)), Aequorin, obelin, or bacterial lux bioluminescence genes from Vibrio fischeri.

5. The method of claim 1, wherein said cell is a melanoma cell.

6. A method of identifying a compound that inhibits MHC II gene expression comprising
a. contacting a test cell with a test compound wherein said cell is comprises:
i. a first nucleic acid comprising a first reporter gene operably linked to an MHC II promoter or fragment thereof; and
ii. a second nucleic acid comprising a second reporter gene operably linked to a non-MHC II promoter
b. comparing the amount expression of said first reporter gene expression to said second reporter gene expression
wherein a decrease of expression of said first reporter gene compared to said second reporter gene indicates that said test compound is an inhibitor of MHC II gene expression.

7. The method of claim 6, wherein said first reporter gene and said second reporter gene are different.

8. The method of claim 6, wherein said non-MHC II promoter is an interferon-γ promoter.

9. The method of claim 6, wherein said cells are further contacted with an inducer compound.

10. The method of claim 8, wherein said inducer compound is interferon-γ.

11. A method of identifying a compound that enhances MHC II gene expression comprising:
a. contacting a test cell with a test compound wherein said cell is transformed with
iii. a first nucleic acid comprising a first reporter gene operably linked to an MHC II promoter or fragment thereof; and
iv. a second nucleic acid comprising a second reporter gene operably linked to a non-MHC II promoter
b. comparing the amount expression of said first reporter gene expression to said second reporter gene expression
wherein an increase of expression of said first reporter gene compared to said second reporter gene indicates that said test compound is an enhancer of MHC II gene expression.

12. A method of identifying a compound that modulates MHC II gene expression comprising:
a. bringing into contact:
i. an MHC II promoter or fragment thereof;
ii. a RFX complex; and
iii. a test compound
under conditions where the MHC II promoter or fragment thereof, and the RFX complex, in the absence of compound, are capable of forming a complex; and
b. comparing the amount of complex formation in the presence of said test compound compared to the absence of said test compound
wherein an increase or a decrease of said complex formation in the presence of said test compound compared to the absence of said test compound indicates that said test compound is a modulator of MHC II gene expression.

13. The method of claim 12, further comprising bringing into contact a CIITA polypeptide under conditions where the MHC II promoter or fragment thereof, the RFX complex and the CIITA polypeptide in the absence of compound, are capable of forming a complex.

14. The method of claim 13, further comprising bringing into contact a X2BP polypeptide and a NF-Y polypeptide under conditions where the MHC II promoter or fragment thereof, the RFX complex, the CIITA polypeptide, the X2BP polypeptide and the NF-Y polypeptide in the absence of compound, are capable of forming a complex.

15. The method of claim 12, wherein said MHC II promoter or fragment thereof is immobilized on a solid support.

16. The method of claim 12, wherein said MHC II promoter further comprises a detectable label.

17. The method of claim 16, wherein said detectable label is streptavidin, biotin, HRP or FITC.

18. The method of claim 13, wherein said CIITA polypeptide is immobilized on a solid support.

19. The method of claim 13, wherein said CIITA polypeptide further comprises a detectable label.

20. The method of claim 12, wherein said RFX complex further comprises a detectable label.

21. The method of claim 12, further comprising one or more wash steps prior to (b).

22. The method of claim 13, further comprising bringing into contact a donor molecule and an acceptor molecule.

23. A method identifying a compound that modulates MHC II gene expression comprising:
a. bringing into contact
i. a first polypeptide selected form the group consisting of a CIITA polypeptide, a RFXANK polypeptide, a RFXAP polypeptide, or a RFX5;
ii. a second polypeptide selected from the group consisting of a RFXANK polypeptide, a RFXAP polypeptide, or a RFX5 polypeptide; and
iii. a test compound
under conditions where said first polypeptide and said second polypeptide, in the absence of compound, are capable of forming a complex; and
b. determining the amount of complex formation
wherein an increase or decrease in the amount of complex formation in the presence of the test compound compared to the absence of the test compound indicates said compound modulates MHC II gene expression.

24. The method of claim 23, wherein said first polypeptide is immobilized on a solid support.

25. The method of claim 23, wherein said second polypeptide further comprises a detectable label.

26. The method of claim 23, further comprising one or more wash steps prior to (b).
